(19) United States
(12) Patent Application Publication
(10) Pub. No.: US 2026/0368912 A1
(43) Pub. Date: Nov. 19, 2026

(54) CO-REGISTRATION OF MULTI-MODALITY VASCULAR IMAGES

(71) Applicant: Boston Scientific Scimed, Inc., Maple Grove, MN (US)

(72) Inventors: Yan Li, Plymouth, MN (US); Hatice Cinar Akakin, Eden Prairie, MN (US); Kevin Bloms, Minneapolis, MN (US); Wenguang Li, Los Gatos, CA (US)

(73) Assignee: Boston Scientific Scimed, Inc., Maple Grove, MN (US)

(21) Appl. No.: 19/674,286

(22) Filed: May 12, 2026

Related U.S. Application Data
(60) Provisional application No. 63/805,422, filed on May 14, 2025.

(51) Int. Cl. G06T 7/33; A61B 8/00; A61B 8/08; A61B 8/12; G06T 11/00

(52) U.S. Cl. CPC G06T 7/337; A61B 8/0891; A61B 8/12; A61B 8/5261; G06T 11/00; G06T 2207/10081; G06T 2207/10132; G06T 2207/30101; G06T 2210/41

(57) ABSTRACT

The present disclosure provides to match landmark features from images of a vascular structure that has been imaged using different imaging modalities. Landmarks features detected from a series of intravascular images can be matched with landmark features detected from an extravascular image. The pairs of matches can be determined based on an initially identified matching of the landmark features and then fine-tune adjusted using one or more image processing techniques.

### CROSS-REFERENCE TO RELATED APPLICATION

This application claims the benefit of U.S. Patent Application Serial No. 63/805,422, filed May 14, 2025, entitled "CO-REGISTRATION OF MULTI-MODALITY VASCULAR IMAGES”, and U.S. Patent Application Serial No. 63/899,461, filed Oct. 15, 2025, entitled "CO-REGISTRATION OF FLUOROSCOPY IMAGES WITH ANGIOGRAPHY AND INTRAVASCULAR ULTRASOUND IMAGES”, which is incorporated by reference herein in its entirety.

### TECHNICAL FIELD

The present disclosure pertains to coronary computed tomographic angiography and intravascular or extravascular imaging modalities and to matching landmarks identified in one imaging modality with landmarks identified in another imaging modality.

### BACKGROUND

Ultrasound devices insertable into a patient's vasculature have proven diagnostic capabilities for a variety of diseases and disorders. For example, intravascular ultrasound (IVUS) imaging systems have been used as an intravascular imaging modality for diagnosing blocked blood vessels and providing information to aid medical practitioners in selecting and placing stents, selecting sites for an atherectomy procedure, or the like.

IVUS imaging systems include a control module (with a pulse generator, an image acquisition and processing components, and a monitor), a catheter, and a transducer disposed in the catheter. The transducer-containing catheter is positioned in a lumen or cavity within, or in proximity to, a region to be imaged, such as a blood vessel wall or patient tissue in proximity to a blood vessel wall. The pulse generator in the control module generates electrical pulses that are delivered to the transducer and transformed to acoustic pulses that are transmitted through patient tissue. The patient tissue (or other structure) reflects the acoustic pulses and reflected pulses are absorbed by the transducer and transformed into electric pulses. The transformed electric pulses are delivered to the image acquisition and processing components and converted into images displayable on the monitor.

### BRIEF SUMMARY

Coronary computed tomographic angiography (CTA or CCTA) is the use of CT imaging modalities to assess the coronary arteries of the heart. Typically, a patient receives an intravenous injection of contrast agent, and then the heart is scanned using a high-speed CT scanner. Coronary CTA and IVUS are often used together to assess coronary vessels. CCTA provides a non-invasive overview of the coronary artery, while IVUS offers detailed insights to diagnose coronary artery disease and evaluate occlusions. Optical Coherence Tomography (OCT) is a sophisticated imaging technique that uses light, in some examples near-infrared light, to create high-resolution images of the coronary arteries.

To aid physicians in reviewing these images, they may be co-registered to each other. For example, each image in a series of IVUS images can be mapped, or co-located, to a position of the vessel represented in the coronary tree extracted from CCTA image. Co-registration of CCTA and IVUS images is a transformative technique that enhances the assessment of coronary artery disease by offering a comprehensive understanding of plaque composition, severity, and vessel anatomy. This advancement significantly improves the precision of stent placement, treatment strategizing, and lesion monitoring. Thus, there is a need for improved co-registration techniques and workflows.

The present disclosure provides a system configured to match landmarks detected in one imaging modality (e.g., IVUS, or the like) with landmarks detected in another, different, imaging modality (e.g., CTA, CCTA, OCT, or the like), and to adjust the positioning of landmarks based on the matching. In general, the present disclosure provides to match the landmarks based on various parameters such as but not limited to side branch locations of vessels extending from a main artery or other imaged vessel. This disclosure provides advancements to the technology of co-registration in that it enables automated co-registration, thereby reducing the need for manual intervention and enhancing the accuracy of the co-registration.

With some embodiments, the disclosure can be implemented as a method for cross modality image co-registration, the method comprising identifying, by a computing device, a first plurality of landmarks in a first image frame corresponding to a first imaging modality; identifying, by the computing device, a second plurality of landmarks in a second image frame corresponding to a second imaging modality different from the first imaging modality; matching, by the computing device, a first start point landmark designated in the first plurality of landmarks in the first image frame to a corresponding second start point landmark feature from the second plurality of landmarks; and co-registering the plurality of second image frames with the first image frame based on the matching, wherein the first imaging modality is coronary computed tomographic angiography and wherein the second imaging modality is intravascular ultrasound.

Alternatively or additionally, embodiments of the method comprise wherein co-registering the plurality of second image frames with the first image frame based on the matching comprises: aligning the first start point landmark designated in the first plurality of landmarks with the corresponding second start point landmark from the second plurality of landmarks; and with the first start point landmark aligned with the second start point landmark, aligning each of a first set of remaining landmarks included in the first plurality of landmarks with a corresponding second set of remaining landmarks included in the second plurality of landmarks using linear mapping.

Alternatively or additionally, embodiments of the method comprise wherein the first start point landmark comprises a landmark corresponding to a side branch junction point where a left anterior descending (LAD) artery couples with a main artery.

Alternatively or additionally, embodiments of the method comprise wherein the first start point landmark comprises a landmark corresponding to a side branch junction point where a left circumflex (LCX) artery couples with a main artery.

Alternatively or additionally, embodiments of the method further comprise: adjusting the alignment between the first set of remaining landmarks and the second set of remaining landmarks by performing an image processing procedure on the alignment of the second set of remaining landmarks using a time warping procedure to adjust the temporal spacing of the landmarks in the second plurality of landmarks.

Alternatively or additionally, embodiments of the method further comprise: wherein at least one landmark of the first plurality of landmarks comprises a distal end point or a proximal end point of a stent located in an imaged portion of a vessel.

Alternatively or additionally, embodiments of the method comprise wherein the at least one landmark of the first plurality of landmarks comprises a side branch junction point indicative of a location of a side vessel extending from a main artery.

Alternatively or additionally, embodiments of the method comprise wherein the at least one landmark of the first plurality of the landmarks comprises a side branch junction point indicative of a location where a circumflex artery extends from a main artery.

Alternatively or additionally, embodiments of the method further comprise: applying a features-based correction algorithm to enhance the matching between the first start point landmark and the corresponding second start point landmark.

Alternatively or additionally, embodiments of the method further comprise: generating a first set of graphical components representing the first image frame with the first plurality of landmarks graphically indicated at their respective locations along a vessel as depicted in the first image frame; generating a second set of graphical components representing the second image frames with the second plurality of landmarks graphically indicated at their respective locations along the vessel as depicted in the second image frames; and generating a graphical image showing the co-registration.

Alternatively or additionally, embodiments of the method comprise wherein generating the graphical image showing co-registration includes displaying the graphical image having the second plurality of landmarks graphically overlaid onto the graphical image of the first image frame.

Alternatively or additionally, embodiments of the method comprise wherein generating the graphical image showing co-registration includes displaying the graphical image comprising: displaying the first image frame with the first plurality of landmarks graphically indicated at the respective locations along a vessel as the vessel is depicted in the first image frame; and displaying the second image frames with the second plurality of landmarks graphically indicated at respective locations along the vessel as the vessel is depicted in the second image frame

Alternatively or additionally, embodiments of the method comprise wherein generating the graphic image showing co-registration includes displaying the graphical image comprising: displaying the first image frame with the first plurality of landmarks graphically indicated at the respective locations along a vessel as the vessel is depicted in the first image frame; and displaying the second image frames with the second plurality of landmarks graphically indicated at respective locations along the vessel as the vessel is depicted in the second image frame, wherein the second image frames and the second plurality of landmarks are displayed in a parallel image along side of the first image frame and the first plurality of landmarks, and arranged so that each of the first plurality of landmarks aligns with a corresponding one of the second plurality of landmarks, wherein corresponding landmarks represent a same physical feature along the vessel as imaged in both the first image frame and in the second image frames.

With some embodiments, the disclosure can be implemented as a computer-readable storage device, the computer-readable storage device comprising instructions executable by a processor of a computing device coupled to an intravascular imaging device and/or a CTA device, wherein when executed the instructions cause the computing device to implement any of the methods disclosed herein.

With some embodiments, the disclosure can be implemented as an apparatus comprising a processor arranged to be coupled to an intravascular imaging device and/or a CT device, the apparatus further comprising a memory comprising instructions, the processor arranged to execute the instructions to implement any of the methods disclosed herein.

With some embodiments, the disclosure can be implemented as an apparatus for a cross-modality image co-registration system, the apparatus comprising: a processor arranged to be coupled to a non-transitory memory storage device, the non-transitory memory storage device comprising computer readable instructions that, when executed by the processor, cause the apparatus to: identify a first plurality of landmarks in a first image frame corresponding to a first imaging modality; identify a second plurality of landmarks in a second image frame corresponding to a second imaging modality different from the first imaging modality; match a first start point landmark designated in the first plurality of landmarks in the first image frame to a corresponding second start point landmark feature from the second plurality of landmarks; and co-register the plurality of second image frames with the first image frame based on the matching, wherein the first imaging modality is coronary computed tomographic angiography and wherein the second imaging modality is intravascular ultrasound.

Alternatively or additionally, embodiments of the apparatus comprise wherein co-registering the plurality of second image frames with the first image frame based on the matching comprises: aligning the first start point landmark designated in the first plurality of landmarks with the corresponding second start point landmark from the second plurality of landmarks; and with the first start point landmark aligned with the second start point landmark, align each of a first set of remaining landmarks included in the first plurality of landmarks with a corresponding second set of remaining landmarks included in the second plurality of landmarks using linear mapping.

Alternatively or additionally, embodiments of the apparatus comprise wherein the first start point landmark comprises a landmark corresponding to a side branch junction point where a left anterior descending (LAD) artery couples with a main artery.

Alternatively or additionally, embodiments of the apparatus comprise wherein the first start point landmark comprises a landmark corresponding to a side branch junction point where a left circumflex (LCX) artery couples with a main artery.

Alternatively or additionally, embodiments of the apparatus further comprise: adjusting the alignment between the first set of remaining landmarks and the second set of remaining landmarks by performing an image processing procedure on the alignment of the second set of remaining landmarks using a time warping procedure to adjust the temporal spacing of the landmarks in the second plurality of landmarks.

Alternatively or additionally, embodiments of the apparatus comprise wherein the at least one landmark of the first plurality of landmarks comprises a side branch junction point indicative of a location of a side vessel extending from a main artery.

Alternatively or additionally, embodiments of the apparatus comprise wherein the at least one landmark of the first plurality of the landmarks comprises a side branch junction point indicative of a location where a circumflex artery extends from a main artery.

Alternatively or additionally, embodiments of the apparatus comprise wherein co-registration further comprises enhancement the co-registration matching accuracy using a features-based correction algorithm.

Alternatively or additionally, embodiments of the apparatus further comprise: generating a first set of graphical components representing the first image frame with the first plurality of landmarks graphically indicated at their respective locations along a vessel as depicted in the first image frame; generating a second set of graphical components representing the second image frames with the second plurality of landmarks graphically indicated at their respective locations along the vessel as depicted in the second image frames; and generating a graphical image showing the co-registration.

Alternatively or additionally, embodiments of the apparatus comprise wherein generating the graphical image showing co-registration includes displaying the graphical image having the second plurality of landmarks graphically overlaid onto the graphical image of the first image frame.

Alternatively or additionally, embodiments of the apparatus comprise wherein generating the graphic image showing co-registration including displaying the graphical image comprises: displaying the first image frame with the first plurality of landmarks graphically indicated at the respective locations along a vessel as the vessel is depicted in the first image frame; and displaying the second image frames with the second plurality of landmarks graphically indicated at respective locations along the vessel as the vessel is depicted in the second image frame, wherein the second image frames and the second plurality of landmarks are displayed in a parallel image alongside of the first image frame and the first plurality of landmarks, and arranged so that each of the first plurality of landmarks aligns with a corresponding one of the second plurality of landmarks, wherein corresponding landmarks represent a same physical feature along the vessel as imaged in both the first image frame and in the second image frames.

With some embodiments, the disclosure can be implemented as a computer-readable storage device comprising instructions executable by a processor of a computing device, wherein the instructions, when executed by the processor cause the computing device to: identify a first plurality of landmarks in a first image frame corresponding to a first imaging modality; identify a second plurality of landmarks in a second image frame corresponding to a second imaging modality different from the first imaging modality; match a first start point landmark designated in the first plurality of landmarks in the first image frame to a corresponding second start point landmark feature from the second plurality of landmarks; and co-register the plurality of second image frames with the first image frame based on the matching, wherein the first imaging modality is coronary computed tomographic angiography and wherein the second imaging modality is intravascular ultrasound.

Alternatively or additionally, embodiments of the non-transitory computer-readable storage device comprise wherein the instructions when executed to match the first start point landmark designated in the first plurality of landmarks in the first image frame to the corresponding second start point landmark feature from the second plurality of landmarks cause the computing device further to: align the first start point landmark designated in the first plurality of landmarks with the corresponding second start point landmark from the second plurality of landmarks; and with the first start point landmark aligned with the second start point landmark, align each of a first set of remaining landmarks included in the first plurality of landmarks with a corresponding second set of remaining landmarks included in the second plurality of landmarks using linear mapping.

Alternatively or additionally, embodiments of the non-transitory computer-readable storage device comprise wherein the first start point landmark comprises a landmark corresponding to a side branch junction point where a left anterior descending (LAD) artery couples with a main artery.

Alternatively or additionally, embodiments of the non-transitory computer-readable storage device comprise wherein the first start point landmark comprises a landmark corresponding to a side branch junction point where a left circumflex (LCX) artery couples with a main artery.

Alternatively or additionally, embodiments of the non-transitory computer-readable storage device comprise wherein the instructions to co-register the plurality of second image frames with the first image frame based on the matching cause the computing device further to: adjust the alignment between the first set of remaining landmarks and the second set of remaining landmarks by performing an image processing procedure on the alignment of the second set of remaining landmarks using a time warping procedure to adjust the temporal spacing of the landmarks in the second plurality of landmarks.

Alternatively or additionally, embodiments of the non-transitory computer-readable storage device comprise wherein the instructions, when executed by the processor, cause the computing device to: generate a first set of graphical components representing the first image frame with the first plurality of landmarks graphically indicated at their respective locations along a vessel as depicted in the first image frame; generate a second set of graphical components representing the second image frames with the second plurality of landmarks graphically indicated at their respective locations along the vessel as depicted in the second image frames; and generate a graphical image showing the co-registration, wherein the generated the graphical image showing co-registration includes displaying the graphical image having the second plurality of landmarks graphically overlaid onto the graphical image of the first image frame.

Alternatively or additionally, embodiments of the non-transitory computer-readable storage device comprise wherein the generated graphical image showing co-registration is generated in real-time while an IVUS image capturing system coupled to the computing device is performing an imaging procedure on a patient.

With some embodiments, the disclosure can be implemented as a method for a cross-modality image co-registration system, the method comprising: identifying, by a computing device, a first plurality of landmarks in a first image frame corresponding to a first imaging modality; identifying, by the computing device, a second plurality of landmarks in a second image frame corresponding to a second imaging modality different from the first imaging modality; matching, by the computing device, a first start point landmark designated in the first plurality of landmarks in the first image frame to a corresponding second start point landmark feature from the second plurality of landmarks; and co-registering the plurality of second image frames with the first image frame based on the matching, wherein the first imaging modality is coronary computed tomographic angiography and wherein the second imaging modality is intravascular ultrasound.

Alternatively or additionally, embodiments of the method comprise wherein co-registering the plurality of second image frames with the first image frame based on the matching comprises: aligning the first start point landmark designated in the first plurality of landmarks with the corresponding second start point landmark from the second plurality of landmarks; and with the first start point landmark aligned with the second start point landmark, aligning each of a first set of remaining landmarks included in the first plurality of landmarks with a corresponding second set of remaining landmarks included in the second plurality of landmarks using linear mapping.

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1 illustrates a multi-modality co-registration system, in accordance with various embodiments.

FIG. 2 illustrates an example first image comprising a three-dimensional coronary artery tree extracted from a CTA image, and an example second image constructed as a long view of a series of IVUS images frames.

FIG. 3 illustrates a graphical depiction of how the landmarks from the IVUS scan image from FIG. 2 can be aligned with the landmarks provided by the coronary artery tree of the CTA image of FIG. 2.

FIG. 4 illustrates an example diagram of co-registration of a CTA image and an IVUS scan image, according to some embodiments of the present disclosure.

FIG. 5 illustrates a routine to co-register IVUS images with a CTA image, according to some embodiments of the present disclosure.

FIG. 6 illustrates a computer-readable storage medium in accordance with at least one embodiment.

FIG. 7 illustrates an example vascular imaging system, in accordance with at least one embodiment.

FIG. 8 illustrates computer-readable storage medium 800, in accordance with some embodiments of the present disclosure.

### DETAILED DESCRIPTION

As noted above, the present disclosure provides systems, methods, and techniques to match landmarks identified from different imaging modalities (e.g., IVUS and angiography). It is noted that although the present disclosure uses examples and describes techniques with reference to IVUS and coronary computed tomographic angiography, any suitable extravascular imaging modality and intravascular imaging modality can be utilized. Further, the present disclosure relies on identified landmarks and their information (e.g., location, size, orientation, etc.). The actual identification of the landmarks themselves is beyond the scope of this disclosure.

Computed Tomography Angiography (CTA) and Intravascular Ultrasound (IVUS) image co-registration is a transformative technique that enhances the assessment of coronary artery disease by providing a comprehensive understanding of plaque composition, severity, and vessel anatomy. This advancement significantly improves the precision of stent placement, treatment planning, and lesion monitoring. This method empowers clinicians to observe dynamic changes in vessel anatomy, plaque characteristics, and stent deployment. The comprehensive feedback offered by this technique facilitates on-the-spot decision-making and allows for real-time adjustments to the procedure for optimized outcomes.

Each imaging modality by itself has limitations and functional features. For example, calcium thickness and density can be extrapolated based on CTA images. However, this information cannot easily be extrapolated from IVUS images alone. One of the main limitations of the IVUS imaging modality, is the lack of calcium thickness information. It is to be appreciated that knowledge of the thickness of calcium is often relied on by physicians in selecting appropriate tools and techniques for calcium modification, such as atherectomy or specialized balloons, to ensure optimal stent deployment. It is to be appreciated that IVUS images provide detailed and high-resolution vessel geometry information. As such, 3D co-registration of CTA images and IVUS image can facilitate the assessment of coronary artery disease by providing a comprehensive understanding of plaque composition, severity, and vessel anatomy.

Given a CTA image, 3D geometry of the vessel and lumen as well as the corresponding arterial tree can be detected and extracted. When IVUS images are captured from within the same vessel, these images (e.g., CTA and IVUS) can be co-registered. For example, landmarks (such as, side branches, circumflex artery, or the like) can be identified from the CTA images and the IVUS images. The landmarks identified from the IVUS images can be mapped to landmarks identified from the CTA image and then the images (e.g., CTA and IVUS) can be co-registered though linear or nonlinear mapping of the mapped landmarks. Further refinements to the co-registration can be applied, for example, to address non-uniform pullback of the IVUS imaging device. It is to be appreciated that the co-registration process can be complicated due to, for example, cardiac motion and the differing resolutions of the imaging modalities being co-registered. Embodiments of the systems, methods, and techniques as described in this disclosure address these issues, to provide accurate and automatically computer-generated co-registration between images comprising imaging data representing two or more different imaging modalities (e.g., CTA and IVUS, or the like).

Besides its clinical value, 3D co-registration of IVUS images with a CTA image holds great potential for research purposes. For example, co-registered sets (also referred to herein as paired images) of IVUS images and a CTA image can be used to implement AI algorithms to predict calcium thickness and density.

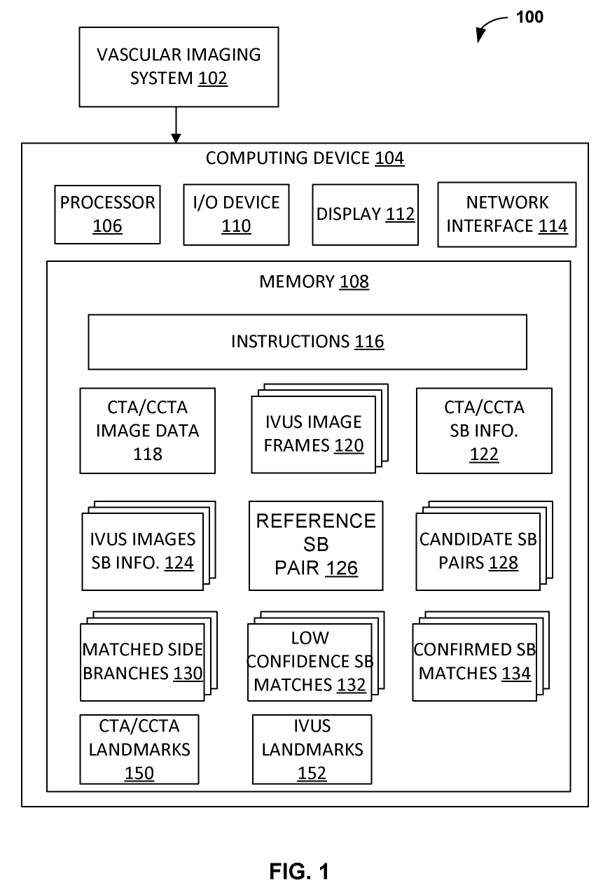

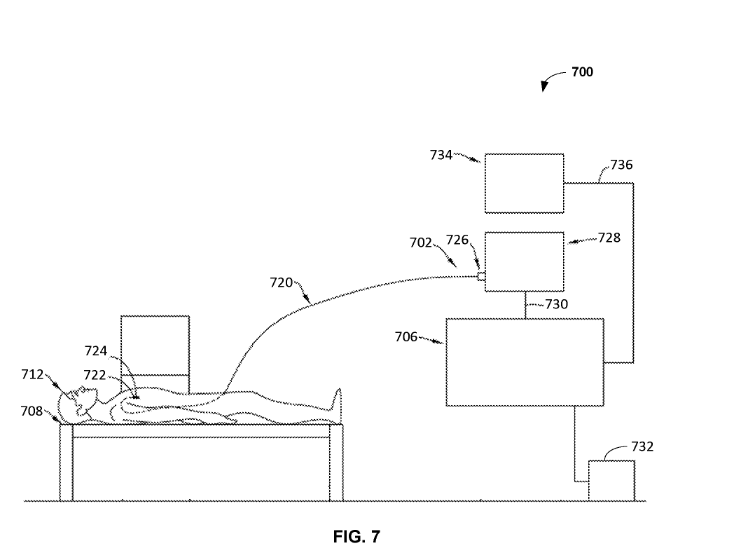

FIG. 1 illustrates a multi-modality co-registration system (system) 100, in accordance with various embodiments of the present disclosure. In general, system 100 is a system configured to identify landmark features, hereinafter “landmarks,” from an extravascular image (e.g., computed tomographic angiography - CTA) and identify corresponding “landmarks” from intravascular images (e.g., IVUS). For example, system 100 can be configured to match landmarks identified from CTA image data 118 with landmarks identified from IVUS image frames 120. To that end, system 100 includes, or can be coupled to, vascular imaging system 102. The vascular imaging system 102 can be any of a variety of vascular imaging systems configured to capture images from multiple imaging modalities (e.g., angiography, CTA, IVUS, intravascular OCT, or the like). An example of a vascular imager configured to capture both external (angiography, CTA) and internal (IVUS) vascular images is described with reference to the combined internal and external imaging system 700 depicted in FIG. 7.

System 100 includes a computing device 104. The computing device 104 can be any of a variety of computing devices. In some embodiments, computing device 104 can be incorporated into and/or implemented by a console of the vascular imaging system 102. With some embodiments, computing device 104 can be a tablet, laptop, workstation, or server communicatively coupled to vascular imaging system 102. With still other embodiments, computing device 104 can be provided by a cloud-based computing device, such as, by a Computing as a Service (CaaS) system accessibly over a network (e.g., the Internet, an intranet, a wide area network, or the like). Computing device 104 can include processor 106, memory 108, input and/or output (I/O) device 110, display 112, and network interface 114.

The processor 106 may include circuity or processor logic, such as, for example, any of a variety of commercial processors. In some examples, processor 106 may include multiple processors, a multi-threaded processor, a multi-core processor (whether the multiple cores coexist on the same or separate dies), and/or a multi-processor architecture of some other variety by which multiple physically separate processors are in some way linked. Additionally, in some examples, the processor 106 may include graphics processing portions and may include dedicated memory, multiple-threaded processing and/or some other parallel processing capability. In some examples, the processor 106 may be an application specific integrated circuit (ASIC) or a field programmable integrated circuit (FPGA).

The memory 108 may include logic, a portion of which includes arrays of integrated circuits, forming non-volatile memory to persistently store data or a combination of non-volatile memory and volatile memory. It is to be appreciated, that the memory 108 may be based on any of a variety of technologies. In particular, the arrays of integrated circuits included in memory 108 may be arranged to form one or more types of memory, such as, for example, dynamic random-access memory (DRAM), NAND memory, NOR memory, or the like.

The I/O devices 110 can be any of a variety of devices to receive input and/or provide output. For example, I/O devices 110 can include, a keyboard, a mouse, a joystick, a foot pedal, a haptic feedback device, an LED, or the like. The display 112 can be a conventional display or a touch-enabled display. Further, display 112 can utilize a variety of display technologies, such as, liquid crystal display (LCD), light emitting diode (LED), or organic light emitting diode (OLED), or the like.

The network interface 114 can include logic and/or features to support a communication interface. For example, network interface 114 may include one or more interfaces that operate according to various communication protocols or standards to communicate over direct or network communication links. Direct communications may occur via use of communication protocols or standards described in one or more industry standards (including progenies and variants). For example, network interface 114 may facilitate communication over a bus, such as, for example, peripheral component interconnect express (PCIe), non-volatile memory express (NVMe), universal serial bus (USB), system management bus (SMBus), SAS (e.g., serial attached small computer system interface (SCSI)) interfaces, serial AT attachment (SATA) interfaces, or the like. Additionally, network interface 114 can include logic and/or features to enable communication over a variety of wired or wireless network standards (e.g., 802.11 communication standards). For example, network interface 114 may be arranged to support wired communication protocols or standards, such as, Ethernet, or the like. As another example, network interface 114 may be arranged to support wireless communication protocols or standards, such as, for example, Wi-Fi, Bluetooth, ZigBee, LTE, 5G, or the like.

Memory 108 can include instructions 116, CTA image data 118, IVUS image frames 120, CTA image side branch (SB) information 122, IVUS images side branch information 124, reference side branch pair 126, candidate side branch pairs 128, matched side branches 130, low confidence side branch matches 132, and confirmed side branch matches 134. Memory 108 may further include CTA landmarks 150 and IVUS landmarks 152. CTA landmarks 150 may include information, such as locations within an CTA image data 118 of a set of landmarks identified from analysis of the angiography image frame. In various embodiments, the CTA landmarks 150 may be identified using side branch information, such as CTA image side branch information 122, which may include 3D coronary artery tree information. IVUS landmarks 152 may include information, such as locations within IVUS image frames 120, of a set of landmarks identified from analysis of the IVUS image frames. In various embodiments, the IVUS landmarks 152 may be identified using side branch information, such as IVUS side branch information 124.

During operation, processor 106 can execute instructions 116 to cause computing device 104 to receive CTA image data 118 and IVUS image frames 120 from vascular imaging system 102. With some examples, processor 106 can execute instructions 116 to cause computing device 104 to receive the CTA image data 118 and the IVUS image frames 120 while they are captured. In other examples, processor 106 can execute instructions 116 to receive the CTA image data 118 or the IVUS image frames 120 while it is captured where the other image modality will have been captured previously. In yet other examples, processor 106 can execute instructions 116 to cause computing device 104 to receive the CTA image data 118 and the IVUS image frames 120, which were both previously captured. In any of these examples, processor 106 can execute instructions 116 to cause computing device 104 to receive data structures of information comprising indications of the CTA image data 118 and IVUS image frames 120 (e.g., from vascular imaging system 102 directly, from network interface 114, from a memory location, or the like).

In general, CTA image data 118 can be a CT image of a patient's heart (or portion of a patient's heart) captured after injection of a contrast agent into the patient's vasculature. With some examples, CTA image data 118 can be a three-dimensional volume image. Similarly, IVUS image frames 120 can be a series of ultrasound images captured from within a vessel of the patient's heart as an ultrasound probe is pulled back through a portion of the vessel. With some embodiments, CT image data 118 can be captured while IVUS image frames 120 is captured. In other embodiments, IVUS image frames 120 can be captured and then CTA image data 118, or vice versa.

As introduced above, the present disclosure provides to “match” landmarks identified from an extravascular image (e.g., CTS image data 118) and intravascular images (e.g., IVUS image frames 120). To that end, CTA image side branch information 122 and 3D coronary artery tree information can be identified from CTA image data 118 and IVUS images side branch information 124 can be identified from IVUS image frames 120. For example, processor 106 can execute instructions 116 to cause computing device 104 to identify CTA image side branch and 3D coronary artery tree information 122 from CTA image data 118 and to identify IVUS images side branch information 124 from IVUS image frames 120. In other examples, CTA image side branch and 3D coronary artery tree information 122 and IVUS images side branch information 124 can be identified by another system and/or computing device and processor 106 can execute instructions 116 to receive (e.g., from network interface 114, from a memory location, or the like) data structures comprising indications of CTA image side branch information 122 and IVUS images side branch information 124. In various embodiments, the CTA image side branch information 122 and IVUS images side branch information 124 can be used as landmarks for the respective CTA image data 118 as CTA landmarks 150 and for IVUS image frames 120 as IVUS landmarks 152.

Although the exact technique and system used to identify CTA image side branch information 122 and 3D coronary artery tree information and IVUS images side branch information 124 is beyond the scope of this disclosure, CTA image side branch and 3D coronary artery tree information 122 and IVUS images side branch information 124 comprise indications of information about the side branches. For example, CTA image side branch and 3D coronary artery tree information 122 can comprise an indication of a location (e.g., two-dimensional (2D) or three-dimensional (3D) location, or the like) of each side branch, defined with respect to the CTA image data 118. Further, CTA image side branch and 3D coronary artery tree information 122 can comprise indications of a size (e.g., in millimeters, or the like) of each side branch as well as three-dimensional location of each side branch. Similarly, IVUS images side branch information 124 can comprise indications of a size and orientation (angular rotation, etc.) of each side branch detected based on the IVUS image frames 120.

The present disclosure provides to “match” the side branches detected based on IVUS image frames 120 with side branches detected based on CTA image data 118 for purposes of co-registration of the images captured by these different image modalities.

In general, processor 106 can execute instructions 116 to cause computing device 104 to identify a side branch from CTA image side branch and 3D coronary artery tree information 122 and a side branch from IVUS images side branch information 124 to designate as a reference side branch pair 126. Further, processor 106 can execute instructions 116 to identify candidate side branch pairs 128 from CTA image side branch information 122 and IVUS images side branch information 124. Candidate side branch pairs 128 comprise several “pairs” of side branches where each pair references a side branch from CTA image side branch information 122 and a side branch from IVUS images side branch information 124. It is noted that some side branches from either CTA image side branch information 122 or IVUS images side branch information 124 may be in multiple “pairs." Said differently, candidate side branch pairs 128 may not define pairs of side branches having a one side branch to 1one side branch (1:1) mapping between CTA image side branch information 122 and IVUS images side branch information 124.

Where candidate side branch pairs 128 comprises a pair that references a side branch in another pair, processor 106 can execute instructions 116 to cause computing device 104 to generate matched side branches 130 based on candidate side branch pairs 128. Where candidate side branch pairs 128 does not comprise a pair, and therefore refences a side branch in another pair, processor 106 can execute instructions 116 to cause computing device 104 to designate candidate side branch pairs 128 and matched side branches 130. Said differently, where the candidate side branch pairs 128 comprises a 1:N (n>1) relationship between paired side branches, for example, where a side branch from IVUS images side branch information 124 is paired with two side branches from CTA image side branch information 122, or vice versa, processor 106 can execute instructions 116 to cause computing device 104 to remove one or more pairs from candidate side branch pairs 128 to generate match side branches 130 having a 1:1 paring (or mapping) between side branches from CTA image side branch information 122 and IVUS images side branch information 134.

Processor 106 can execute instructions 116 to cause computing device 104 to identify low confidence side branch matches 132 and generate confirmed side branch matches 134 based in part on matched side branches 130 and low confidence side branch matches 132. For example, pairs identified in matched side branches 130 with a confidence value below a threshold can be removed from matched side branches 130, resulting in confirmed side branch matches 1. In various embodiments, a non-limiting example of a confidence threshold value.

In various embodiments, features in CTA image data 118 and IVUS image frames 120 other than side branch features may be processed and identified as landmarks in the images. For example, features such as a distal end point of a stent located in the imaged portion of a vessel, and/or a proximal end point of the stent located in the imaged portion of the vessel, may be identified in both the CTA image data 118 and IVUS image frames 120, and utilized as CTA landmarks 150 and IVUS landmarks 152, respectively. Other features, such as plaque and/or lesions present in the imaged portion of the vessel may be utilized as landmarks in CTA landmarks 150 and IVUS landmarks 152, respectively. In other words, any feature that can be identified in one of both the image data provided by CTA image data 118 and/or by IVUS image frames 120 may be considered for use as landmarks in one or more of CTA landmarks 150 and IVUS landmarks 152.

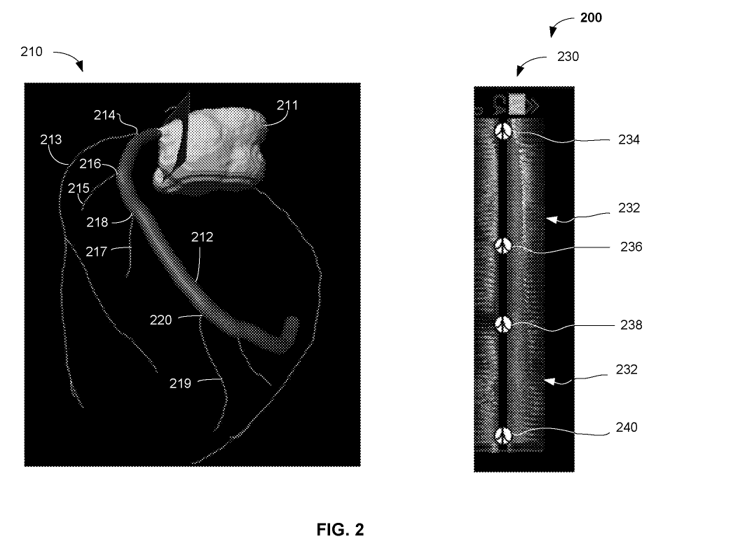

FIG. 2 illustrates a diagram 200 of an example first (CTA 3D coronary artery tree) image 210 comprising a three-dimensional image based on a CTA image data, and an example second (IVUS) image 230 constructed as a long view of a series of IVUS images frames 232. In first image 210, a main vessel 212 extends from a heart 211 along a length that includes side branch junction point 214 coupling a left anterior descending (LAD) artery 213 to the main vessel 212. Additional side branch vessels are coupled to the main vessel 212, including side branch vessel 215 coupled to main vessel 212 at side branch junction point 216, side branch vessel 217 coupled to main vessel 212 at side branch junction point 218, and side branch vessel 219 coupled to main vessel 212 at side branch junction point 220. Side branch junction points 214, 216, 218, and 220 are spaced apart from one another along the main vessel 212 extending in a direction away from heart 211. In various embodiments, the LDA side branch junction point 214 and each of additional side branch junction points 216, 218, and 220 may be constructed as landmark features, or simply referred to as “landmarks” for the first image 210 and may be stored in memory 108 (FIG. 1) as CTA landmarks 150 (FIG. 1).

As also shown in FIG. 2, a corresponding IVUS image 230 may include a temporal compilation of a series of IVUS image frames 232 that were images along a length of the same main vessel 212 shown in the 3D coronary artery tree image based on CTA image 210. The series of IVUS image frames 232 are depicted as complied images forming a long view of main vessel 212, and extend from just beyond the corresponding LDA side branch junction point 214 in CTA image 210, labeled as side branch junction point 234 in the IVUS image 230, to just beyond the side branch junction point 220 in CTA image 210, which is labeled as side branch junction point 240 in the IVUS image 230. The IVUS image 230 also includes labels indicating the positioning of side branch junction point 236 and side branch junction point 238, which correspond to side branch junction points 216 and 218, respectively, in the CTA image 210.

In various embodiments, the side branch junction points 234, 236, 238, and 240 may be constructed as landmark features, or simply referred to as “landmarks” for the IVUS scan image 230 and for example stored in memory 108 (FIG. 1) as IVUS landmarks 152 (FIG. 1). Once the landmark features or “landmarks” have been identified for both the CTA image 210 and the IVUS image 230, the IVUS image 230 can be co-registered with the CTA image 210 though linear or non-linear mapping. Co-registering CTA image 210 and the IVUS scan image 230 involves aligning the two modalities to accurately match anatomical features.

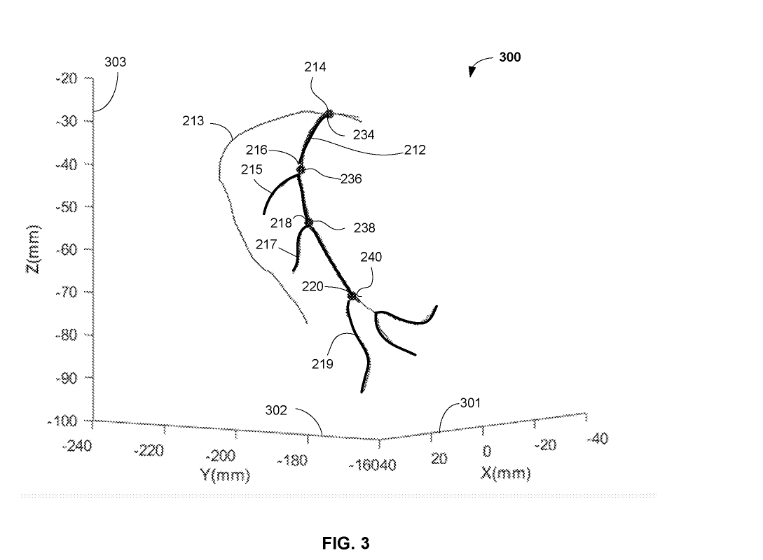

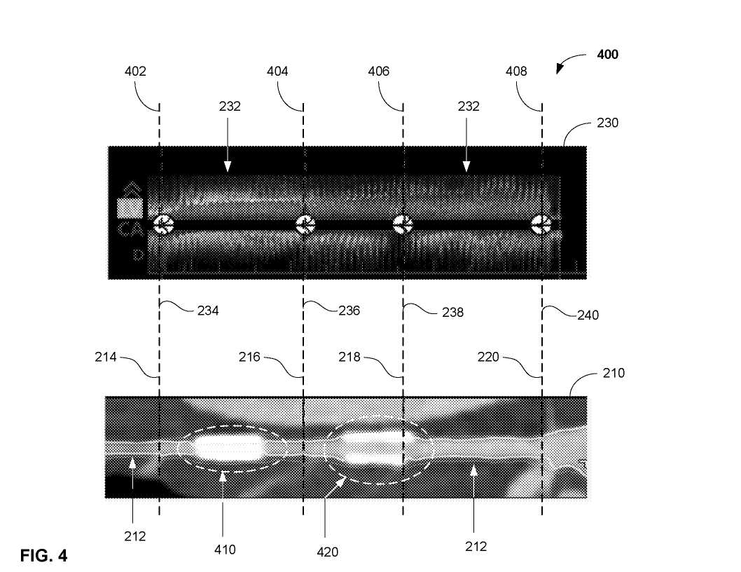

FIG. 3 illustrates a graphical depiction 300 of how the landmarks from the IVUS image 230 from FIG. 2 can be aligned with the landmarks provided by the CTA image 210 of FIG. 2. FIG. 4 illustrates additional details about the alignment between the IVUS image and 3D coronary artery tree of the CTA image 210, and refining adjustments that may be performed on the co-registered images to adjust for issues such as the pullback variations caused by the IVUS imaging process performed to image the main vessel 212 in order to generate the IVUS image frames 232.

As shown in FIG. 3, graphical depiction 300 includes a depiction of IVUS image 230 co-registered and overlaid on top of a depiction of 3D coronary artery tree of the CTA image 210. IVUS image 230 includes image frames 232 that extend along main vessel 212 from just beyond side branch junction point 234 through the main vessel 212 to just beyond side branch junction point 240 and including side branch junction points 236 and 238 located at separate and different locations along main vessel 212 and between side branch junction points 234 and 240. Each of the side branch junction points 234, 236, 238, and 240 may be considered as landmarks included in IVUS landmarks 152 identified for IVUS image 230.

As also shown in FIG. 3, graphical depiction 300 includes a depiction of 3D coronary artery three of the CTA image 210, including main vessel 212, and side branches 213, 215, 217, and 219 extending from main vessel 212 as side branch junction points 214, 216, 218, and 220, respectively, positioned and separate and different locations along main vessel 212. Each of the side branch junction points 214, 216, 218, and 220 may be considered as landmarks included in CTA landmarks 150 identified for 3D coronary artery tree of the CTA image 210.

When overlaid as shown in graphical depiction 300, the landmarks from IVUS image 230 may be rendered to overlay the corresponding landmarks identified in 3D coronary artery tree of the CTA image 210. For example, in graphical depiction 300, side branch junction point 234 from IVUS image 230 is overlaid onto corresponding side branch junction point 214 identified as the corresponding landmark in 3D coronary artery tree of the CTA image 210. Using the overlay of side branch junction point 234 from IVUS image 230 onto corresponding side branch junction point 214 as a starting point, the remaining landmarks as identified in IVUS image 230 may be fitted along the image of main vessel 212 so that each of the remaining landmarks, i.e., side branch junction points 216, 218, and 220 from IVUS image 230 are overlaid onto the respective corresponding side branch junction points 236, 238, and 240 from CTA image 210. In various embodiments, linear mapping and/or other image processing techniques may be used to position and shape the image frames 232 form IVUS image 230 into the three-dimensional configuration provided in the 3D coronary artery tree of the CTA image 230.

Further refinement, as further discussed below, can be applied to address potential non-uniformity between the overlaid IVUS image 230 and the 3D coronary artery tree of the CTA image 210. Issues that could potentially create non-uniformity between the images can include but are not limited to minor variations in the positioning and the routing of the main vessel caused by the pullback actions of IVUS probe occurring during the IVUS imaging process, and/or due to cardiac motions that may occur during both the IVUS imaging process and the CTA imaging process. Further, the process can be complicated due to differences between the resolutions of the different imaging modalities.

As such, co-registering CTA and IVUS images involving aligning the two modalities to accurately match anatomical features is challenging. The systems, methods, and techniques as described herein address these issues through the use of anatomical landmarks to identify either start or end points for initial alignment through linear mapping. Additionally, other landmarks can be used for fine adjustments to enhance co-registration accuracy. For further enhancement, dynamic time warping and feature-based correction algorithms can be implemented to achieve fine alignment between the aligned landmarks. Additionally, other landmarks can be used for fine adjustments to enhance co-registration accuracy.

The capability to accurately co-register images generated using two different or multiple different modalities represents an improvement to the technical field of vascular imaging. The capability to accurately co-register images from different image modalities as described herein is a transformative technique that enhances the assessment of coronary artery disease by offering a more comprehensive understanding of plaque composition, severity, and vessel anatomy. In various embodiments, the results of the co-registration techniques as applied to render these different modality images into a common image, including for example as rendered into the three-dimensional image as shown in FIG. 3, having X-axis 301, Y-axis 302, and Z-axis 303 dimensional aspects, may be displayed as a graphical user interface (GUI) on a display device, such as display 112 (FIG. 1). The availability of this information provided by the co-registration method and techniques as described herein represent a practical application and an advancement that can significantly improve cardiac care including but not limited to the precision of stent placement, treatment strategizing, and lesion monitoring.

FIG. 4 illustrates an example diagram 400 of co-registration of a CTA image and an IVUS image, according to various embodiments. Diagram 400 includes IVUS image 230 (FIG. 2) positioned in a horizontal orientation and above CTA image 210, (also from FIG. 2), which is also oriented in a horizontal orientation. The left-to-right positioning of IVUS image 230 is aligned with the left-to-right position of CTA image 210 such that each of the identified landmarks in IVUS image 230 aligns vertically with a respective and corresponding landmark identified in CTA image 210.

As shown in diagram 400: landmark 234 in IVUS image 230 aligns with vertical dashed line 402, which also aligns with landmark 214 in CTA image 210, landmark 236 in IVUS image 230 aligns with vertical dashed line 404, which also aligns with landmark 216 in CTA image 210, landmark 238 in IVUS scan image 230 aligns with vertical dashed line 406, which also aligns with landmark 218 in CTA image 210, and landmark 240 in IVUS image 230 aligns with vertical dashed line 408, which also aligns with landmark 220 in CTA image 210.

In various embodiments, the co-registration of CTA image 210 and IVUS scan image 230 may be an initial alignment using only a matchup of the corresponding identified landmarks for each of the images. Fine adjustment(s) to more accurately align the two images may then be performed. For example, time warping may be applied to adjust the temporal spacing to the image frames included in the IVUS image 230, thereby adjusting the alignment between features in the IVUS image 230 more accurately to the corresponding features in the CTA image 210.

In various embodiments, advantages for the co-registration of the IVUS image 230 and the CTA image 210 include the ability to locate features, such as calcium deposits and/or stents that may be present in the vessel and which are not as easily discernable in the IVUS image 230 as compared to the CTA image 210. For example, in diagram 400 the area of main vessel 212 enclosed by dashed ellipse 410 represents a calcium deposit located within the vessel. The indication of the calcium is evident in the CTA image 210, but that may be difficult to discern from a visual perspective in the IVUS scan image 230. In another example, in diagram 400 the area of main vessel 212 enclosed by dashed ellipse 420 represents a stent that is located within the vessel. The indication of the stent is evident in the CTA image 210, but that may be difficult to discern from a visual perspective in the IVUS scan image 230. Because of the co-registration provided in diagram 400, the area within the image frames 232 that correspond to the area where the calcium is located within the vessel is easily determined by the layout and co-registration provided by diagram 400. Because of the co-registration provided in diagram 400, the area within the image frames 232 that correspond to the area where the stent is located within the vessel is easily determined by the layout and co-registration provided by diagram 400.

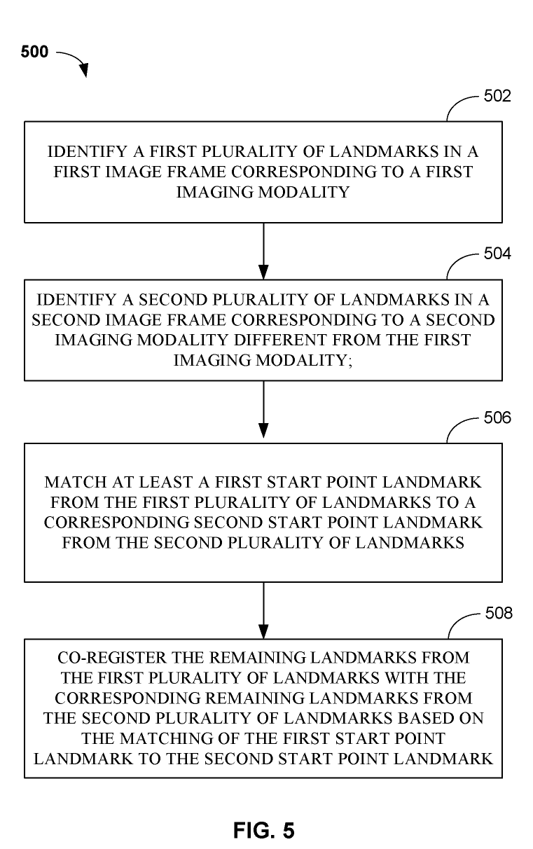

FIG. 5 illustrates routine 500, which can be implemented by system 100, or another computing device, as outlined herein to co-register the CTA generated image with IVUS generated images based on landmarks identified for each of the images.

Routine 500 can begin at block 502 “identify a first plurality of landmarks in a first image frame corresponding to a first imaging modality,” which can include receiving, at a computing device, image data captured using a first imaging modality, which in some embodiments comprises a CTA image captured using CTA equipment. For example, processor 106 can execute instructions 116 to cause computing device 104 to receive CTA image data 118, as well as CTA image side branch information 122. In some examples, processor 106 can execute instructions 116 at block 202 to cause computing device 104 to also receive CTA image data 118, and to process the received CTA image data 118 to generate CTA landmarks 150, comprising identifying the locations of a first plurality of landmark features in the received CTA image data 118. The methods and/or techniques utilized to detect and determine the respective location of the landmark features is not limited to any particular method(s) and/or to any particular technique(s), and may include the use of machine learning, neural network(s), artificial intelligence tools, or some combination thereof.

Routine 500 can continue at block 504, “identify a second plurality of landmarks in a second image frame corresponding to a second imaging modality different from the first imaging modality,” which can include receiving, at a computing device 104, image data captured using a second imaging modality, which in some embodiments comprises a series of IVUS image frames captured using IVUS imaging equipment. For example, processor 106 can execute instructions 116 to cause computing device 104 to receive IVUS image frames 120. In some examples, processor 106 can execute instructions 116 at block 504 to cause computing device 104 to process the received IVUS image frames 120 to generate IVUS landmarks 152, comprising identifying the locations of a second plurality of landmark features based on the received IVUS image frames 120. The methods and/or techniques utilized to detect and determine the respective locations of the landmark features in the IVUS Image frames 120 is not limited to any particular method(s) and/or to any particular technique(s), and may include the use of machine learning, neural network(s), artificial intelligence tools, or some combination thereof.

With the first plurality of landmarks identified as CTA landmarks 150 from the CTA image data 118, and the second plurality of landmarks identified as IVUS landmarks 152 based on the processing performed on the IVUS image frames 120, routine 500 may proceed to block 506, “match at least a first start point landmark from the first plurality of landmarks to a corresponding second start point landmark from the second plurality of landmarks from the second imaging modality.” For example, processor 106 can execute instructions 116 to cause computing device 104 to determine a first start point landmark from the first plurality of landmarks based on a predefined landmark, in some embodiments a landmark indicative of a particular side branch location within the first image frame. In various embodiments, the first start point landmark may be a side branch that is easily identified from the CTA image data 118, such as landmark 214 where the side branch where the LDA joins the main vessel 212. In addition, processor 106 can execute instructions 116 to cause computing device 104 to determine a corresponding second start point landmark from the second plurality of landmarks based on matching a landmark from the second plurality of landmarks that corresponds to a same physical feature indicated by the first start point landmark from the first plurality of landmarks, which again in some embodiments is a landmark indicative of a particular side branch location within the second image frame. In various embodiments, when the first start point landmark is a landmark associated with side branch junction point, such as landmark 214 where the side branch where the LDA joins the main artery in CTA image 210 (FIGS. 2–4), the corresponding second starting point landmark will be landmark 234 (FIGS. 2–4), corresponding to the physical location where the LDA vessel coupled with the depiction of main vessel 232 in the IVUS image frames 230.

Once a match between the first start point landmark and the corresponding second start point landmark has been made at block 506, routine 500 continues at block 508, “co-register the remaining landmarks from first plurality of landmarks with the corresponding remaining landmarks from the second plurality of landmarks based on the matching of the first start point landmark to the second start point landmark.” In various embodiments, the “remaining landmarks” from the first plurality of landmarks comprise any of the landmarks identified for the first image frame (CTA landmarks 150, FIG. 1), but not including the landmark determined to be a first start point landmark. Similarly, the “remaining landmarks” from the second plurality of landmarks comprise any of the landmarks identified for the second image frame (IVUS landmarks 152, FIG. 1), but not including the landmark determined to be a second start point landmark.

In various embodiments, processor 106 can execute instructions 116 to cause computing device 104 to align one, some, or all of the remaining landmarks for first plurality of landmarks with a corresponding and single one of the remaining landmarks from the second plurality of landmarks to generate co-registration between the first image frame and the second image frame. In various embodiments, the initial co-registration between the first image frame and the second image frame may be performed using linear and/or nonlinear mapping and/or feature recognition software. After completion of the initial co-registration, processor 106 can execute instructions 116 to cause computing device 104 to make refining adjustments to the co-registrations of the first image frame and the second image frame to enhance co-registration accuracy. For further enhancement, embodiments can include use of dynamic time warping and feature-based correction algorithms to achieve fine alignment between the initially aligned landmarks.

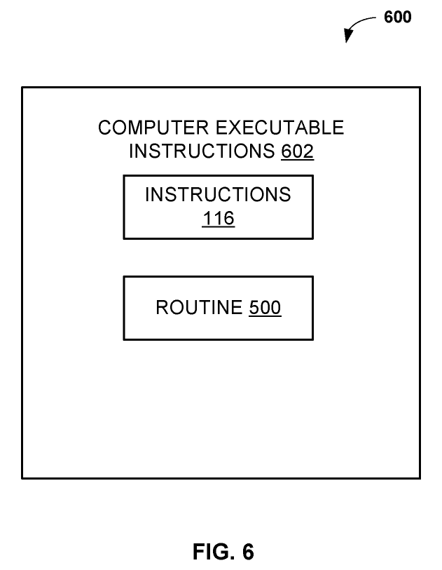

FIG. 6 illustrates computer-readable storage medium 600. Computer-readable storage medium 600 may comprise any non-transitory computer-readable storage medium or machine-readable storage medium, such as an optical, magnetic or semiconductor storage medium. In various embodiments, computer-readable storage medium 600 may comprise an article of manufacture. In some embodiments, computer-readable storage medium 600 may store computer executable instructions 602 with which circuitry (e.g., processor 106, or the like) can execute. For example, computer executable instructions 602 can include instructions to implement operations described with respect to system 100, which can improve the functioning of system 100 as detailed herein. For example, computer executable instructions 602 can include instructions that can cause a computing device to implement routine 500 of FIG. 5. As another example, computer executable instructions 602 can include instructions 116. Examples of computer-readable storage medium 600 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 602 may include any suitable type of code, such as source code, compiled code, interpreted code, executable code, static code, dynamic code, object-oriented code, visual code, and the like.

FIG. 7 illustrates an imaging system 700 including both an endoluminal imaging system 702 (e.g., an IVUS imaging system, or the like). Embodiments of imaging system 700 include computing device 706, which includes circuitry, controllers, and/or processor(s) and memory and software as needed. With some embodiments, system 100 (FIG. 1) can be incorporated into computing device 706, or system 100 can incorporate computing device 706. In general, the endoluminal imaging system 702 can be arranged to generate intravascular imaging data (e.g., IVUS images, IVUS image frames 120, or the like). In various embodiments, computing device 706 may already include data, such as CTA image data 118 (FIG. 1) which may have previously obtained using a separate extravascular imaging system (not shown in FIG. 7) with respect to the same patient 712 now undergoing for example the IVUS imaging procedure as depicted in FIG. 7.

The imaging sytem 700 may include a table 708 that may be arranged to provide sufficient space for the positioning a patient 712. The intravascular imaging data may be, for example, IVUS data or OCT data obtained by the endoluminal imaging system 702. The endoluminal imaging system 702 may include an intravascular imaging device such as an imaging catheter 720. The imaging catheter 720 is configured to be inserted within the patient 712 so that its distal end, including a diagnostic assembly or probe 722 (e.g., an IVUS probe), is in the vicinity of a desired imaging location of a blood vessel. A radiopaque material or marker 724 located on or near the probe 722 may provide indicia of a current location of the probe 722 in a radiological image. In some embodiments, imaging catheter 720 and/or probe 722 can include a guide catheter (not shown) that has been inserted into a lumen of the subject (e.g., a blood vessel, such as a coronary artery) over a guidewire (also not shown). However, in some embodiments, the imaging catheter 720 and/or probe 722 can be inserted into the vessel of the patient 712 without a guidewire.

With some embodiments, imaging catheter 720 and/or probe 722 can include both imaging capabilities as well as other data-acquisition capabilities. For example, FFR and/or iFR data, data related to pressure, flow, temperature, electrical activity, oxygenation, biochemical composition, or any combination thereof. In some embodiments, imaging catheter 720 and/or probe 722 can further include a therapeutic device, such as a stent, a balloon (e.g., an angioplasty balloon), a graft, a filter, a valve, and/or a different type of therapeutic endoluminal device.

Imaging catheter 720 is coupled to a proximal connector 726 to couple imaging catheter 720 to image acquisition device 728. Image acquisition device 728 may be coupled to computing device 706 via transmission cable, or a wireless connection. The intravascular image data may be initially stored in memory within the image acquisition device 728 or may be stored within memory of computing device 706. If the image acquisition device 728 is a separate component from computing device 706, the intravascular image data may be transferred to the computing device 706, via, for example, transmission cable 730.

The computing device 706 can also include one or more additional output ports for transferring data to other devices. For example, the computing device 706 can include an output port to transfer data to a data archive or memory device 732. The computing device 706 can also include a user interface (described in greater detail below) that includes a combination of circuitry, processing components and instructions executable by the processing components and/or circuitry to enable the image identification and vessel routing or pathfinding described herein and/or dynamic co-registration of intravascular and extravascular images using the identified vessel pathway. In some embodiments, computing device 706 can include user interface devices, such as, a keyboard, a mouse, a joystick, a touchscreen device (such as a smartphone or a tablet computer), a touchpad, a trackball, a voice-command interface, and/or other types of user interfaces that are known in the art.

The user interface can be rendered and displayed on display 734 coupled to computing device 706 via display cable 736. Although the display 734 is depicted as separate from computing device 706, in some examples the display 734 can be part of computing device 706. Alternatively, the display 734 can be remote and wireless from computing device 706. As another example, the display 734 can be part of another computing device different from computing device 706, such as, a tablet computer, which can be coupled to computing device 706 via a wired or wireless connection. For some applications, the display 734 includes a heads-up display and/or a head-mounted display. For some applications, the computing device 706 generates an output on a different type of visual, text, graphics, tactile, audio, and/or video output device, e.g., speakers, headphones, a smartphone, or a tablet computer. For some applications, the user interface rendered on display 734 acts as both an input device and an output device.

In various embodiments, display 734 may be configured to provide graphical displays showing the co-registrations of two different imaging modalities, such as co-registration of images generated by endoluminal imaging system 702 (e.g., an IVUS imaging system, or the like) and CTA image data, (e.g., an CTA image data 118, previously captured by a CTA imaging system), wherein the co-registration of the images was performed using landmarks identified in each of the image modalities using any of the methods and techniques as described in this disclosure, and any equivalents thereof. In various embodiments, the co-registration of the images may be performed in real-time or near real time as the IVUS imaging is being performed so that the IVUS images being captured at the time may be co-registered with CTA image data previously captured and displayed, for example on display 734, in real time or near real time as the IVUS imaging procedure is being performed on patient 712.

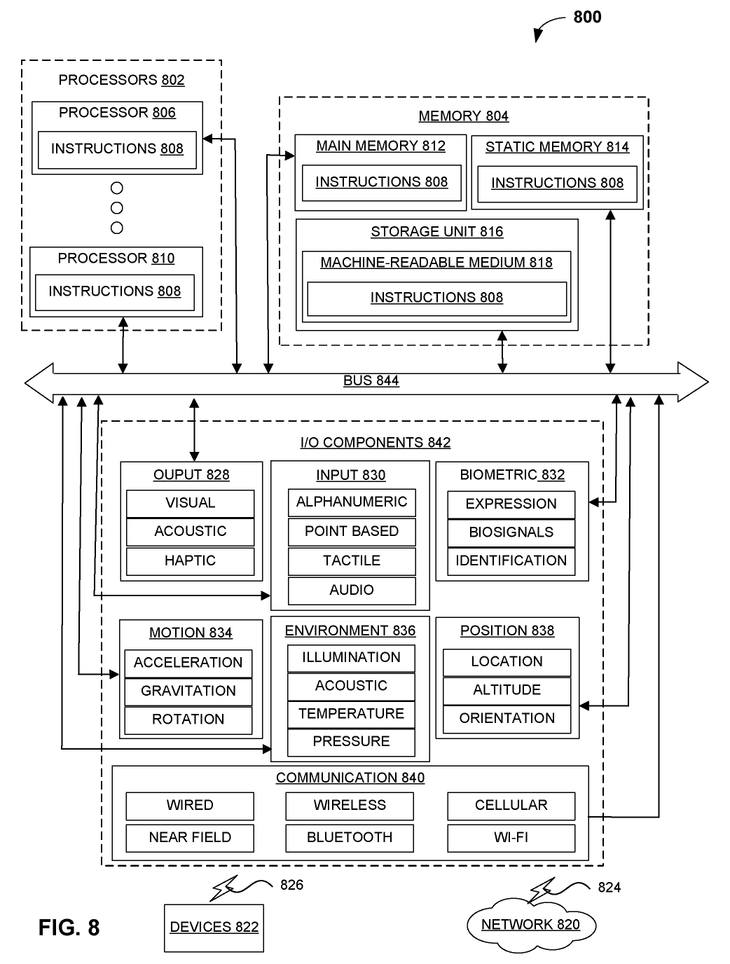

FIG. 8 illustrates a diagrammatic representation of a machine 800 in the form of a computer system within which a set of instructions may be executed for causing the machine to perform any one or more of the methodologies discussed herein. More specifically, FIG. 8 shows a diagrammatic representation of the machine 800 in the example form of a computer system, within which instructions 808 (e.g., software, a program, an application, an applet, an app, or other executable code) for causing the machine 800 to perform any one or more of the methodologies discussed herein may be executed. For example, the instructions 808 may cause the machine 800 to execute instructions 116, or routine 500 of FIG. 5, or the like. More generally, the instructions 808 may cause the machine 800 to co-register IVUS and angiography images as outlined herein.

The instructions 808 transform the general, non-programmed machine 800 into a particular machine 800 programmed to carry out the described and illustrated functions in a specific manner. In alternative embodiments, the machine 800 operates as a standalone device or may be coupled (e.g., networked) to other machines. In a networked deployment, the machine 800 may operate in the capacity of a server machine or a client machine in a server-client network environment, or as a peer machine in a peer-to-peer (or distributed) network environment. The machine 800 may comprise, but not be limited to, a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 808, sequentially or otherwise, that specify actions to be taken by the machine 800. Further, while only a single machine 800 is illustrated, the term “machine” shall also be taken to include a collection of machines 800 that individually or jointly execute the instructions 808 to perform any one or more of the methodologies discussed herein.

The machine 800 may include processors 802, memory 804, and I/O components 842, which may be configured to communicate with each other such as via a bus 844. In an example embodiment, the processors 802 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) may include, for example, a processor 806 and a processor 810 that may execute the instructions 808. The term “processor” is intended to include multi-core processors that may comprise two or more independent processors (sometimes referred to as “cores”) that may execute instructions contemporaneously. Although FIG. 8 shows multiple processors 802, the machine 800 may include a single processor with a single core, a single processor with multiple cores (e.g., a multi-core processor), multiple processors with a single core, multiple processors with multiples cores, or any combination thereof.

The memory 804 may include a main memory 812, a static memory 814, and a storage unit 816, both accessible to the processors 802 such as via the bus 844. The main memory 804, the static memory 814, and storage unit 816 store the instructions 808 embodying any one or more of the methodologies or functions described herein. The instructions 808 may also reside, completely or partially, within the main memory 812, within the static memory 814, within machine-readable medium 818 within the storage unit 816, within at least one of the processors 802 (e.g., within the processor’s cache memory), or any suitable combination thereof, during execution thereof by the machine 800.

The I/O components 842 may include a wide variety of components to receive input, provide output, produce output, transmit information, exchange information, capture measurements, and so on. The specific I/O components 842 that are included in a particular machine will depend on the type of machine. For example, portable machines such as mobile phones will likely include a touch input device or other such input mechanisms, while a headless server machine will likely not include such a touch input device. It will be appreciated that the I/O components 842 may include many other components that are not shown in FIG. 8. The I/O components 842 are grouped according to functionality merely for simplifying the following discussion and the grouping is in no way limiting. In various example embodiments, the I/O components 842 may include output components 828 and input components 830. The output components 828 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 830 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further example embodiments, the I/O components 842 may include biometric components 832, motion components 834, environmental components 836, or position components 838, among a wide array of other components. For example, the biometric components 832 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 834 may include acceleration sensor components (e.g., accelerometer), gravitation sensor components, rotation sensor components (e.g., gyroscope), and so forth. The environmental components 836 may include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 838 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 842 may include communication components 840 operable to couple the machine 800 to a network 820 or devices 822 via a coupling 824 and a coupling 826, respectively. For example, the communication components 840 may include a network interface component or another suitable device to interface with the network 820. In further examples, the communication components 840 may include wired communication components, wireless communication components, cellular communication components, Near Field Communication (NFC) components, Bluetooth^{®} components (e.g., Bluetooth^{®} Low Energy), Wi-Fi^{®} components, and other communication components to provide communication via other modalities. The devices 822 may be another machine or any of a wide variety of peripheral devices (e.g., a peripheral device coupled via a USB).

Moreover, the communication components 840 may detect identifiers or include components operable to detect identifiers. For example, the communication components 840 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 840, such as location via Internet Protocol (IP) geolocation, location via Wi-Fi® signal triangulation, location via detecting an NFC beacon signal that may indicate a particular location, and so forth.

The various memories (i.e., memory 804, main memory 812, static memory 814, and/or memory of the processors 802) and/or storage unit 816 may store one or more sets of instructions and data structures (e.g., software) embodying or utilized by any one or more of the methodologies or functions described herein. These instructions (e.g., the instructions 808), when executed by processors 802, cause various operations to implement the disclosed embodiments.

As used herein, the terms “machine-storage medium,” “device-storage medium,” “computer-storage medium” mean the same thing and may be used interchangeably in this disclosure. The terms refer to a single or multiple storage devices and/or media (e.g., a centralized or distributed database, and/or associated caches and servers) that store executable instructions and/or data. The terms shall accordingly be taken to include, but not be limited to, solid-state memories, and optical and magnetic media, including memory internal or external to processors. Specific examples of machine-storage media, computer-storage media and/or device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms “machine-storage media,” “computer-storage media,” and “device-storage media” specifically exclude carrier waves, modulated data signals, and other such media, at least some of which are covered under the term “signal medium” discussed below.

In various example embodiments, one or more portions of the network 820 may be an ad hoc network, an intranet, an extranet, a VPN, a LAN, a WLAN, a WAN, a WWAN, a MAN, the Internet, a portion of the Internet, a portion of the PSTN, a plain old telephone service (POTS) network, a cellular telephone network, a wireless network, a Wi-Fi® network, another type of network, or a combination of two or more such networks. For example, the network 820 or a portion of the network 820 may include a wireless or cellular network, and the coupling 824 may be a Code Division Multiple Access (CDMA) connection, a Global System for Mobile communications (GSM) connection, or another type of cellular or wireless coupling. In this example, the coupling 824 may implement any of a variety of types of data transfer technology, such as Single Carrier Radio Transmission Technology (1xRTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long range protocols, or other data transfer technology.

The instructions 808 may be transmitted or received over the network 820 using a transmission medium via a network interface device (e.g., a network interface component included in the communication components 840) and utilizing any one of several well-known transfer protocols (e.g., hypertext transfer protocol (HTTP)). Similarly, the instructions 808 may be transmitted or received using a transmission medium via the coupling 826 (e.g., a peer-to-peer coupling) to the devices 822. The terms “transmission medium” and “signal medium” mean the same thing and may be used interchangeably in this disclosure. The terms “transmission medium” and “signal medium” shall be taken to include any intangible medium that can store, encoding, or carrying the instructions 808 for execution by the machine 800, and includes digital or analog communications signals or other intangible media to facilitate communication of such software. Hence, the terms “transmission medium” and “signal medium” shall be taken to include any form of modulated data signal, carrier wave, and so forth. The term “modulated data signal” means a signal that has one or more of its characteristics set or changed in such a matter as to encode information in the signal.

Terms used herein should be accorded their ordinary meaning in the relevant arts, or the meaning indicated by their use in context, but if an express definition is provided, that meaning controls.

Herein, references to "one embodiment" or "an embodiment" do not necessarily refer to the same embodiment, although they may. Unless the context clearly requires otherwise, throughout the description and the claims, the words "comprise," "comprising," and the like are to be construed in an inclusive sense as opposed to an exclusive or exhaustive sense; that is to say, in the sense of "including, but not limited to." Words using the singular or plural number also include the plural or singular number respectively, unless expressly limited to one or multiple ones. Additionally, the words "herein," "above," "below" and words of similar import, when used in this application, refer to this application as a whole and not to any portions of this application. When the claims use the word "or" in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

What is claimed is:

1. An apparatus for a cross-modality image co-registration system, comprising:
a processor arranged to be coupled to a non-transitory memory storage device, the non-transitory memory storage device comprising computer readable instructions that, when executed by the processor, cause the apparatus to:
identify a first plurality of landmarks in a first image frame corresponding to a first imaging modality;
identify a second plurality of landmarks in a second image frame corresponding to a second imaging modality different from the first imaging modality;
match a first start point landmark designated in the first plurality of landmarks in the first image frame to a corresponding second start point landmark feature from the second plurality of landmarks; and
co-register the plurality of second image frames with the first image frame based on the matching, wherein the first imaging modality is coronary computed tomographic angiography and wherein the second imaging modality is intravascular ultrasound.

2. The apparatus of claim 1, wherein co-registering the plurality of second image frames with the first image frame based on the matching comprises:
aligning the first start point landmark designated in the first plurality of landmarks with the corresponding second start point landmark from the second plurality of landmarks; and
with the first start point landmark aligned with the second start point landmark, align each of a first set of remaining landmarks included in the first plurality of landmarks with a corresponding second set of remaining landmarks included in the second plurality of landmarks using linear mapping.

3. The apparatus of claim 1, wherein the first start point landmark comprises a landmark corresponding to a side branch junction point where a left anterior descending (LAD) artery couples with a main artery.

4. The apparatus of claim 1, wherein the first start point landmark comprises a landmark corresponding to a side branch junction point where a left circumflex (LCX) artery couples with a main artery.

5. The apparatus of claim 1, further comprising:
adjusting the alignment between the first set of remaining landmarks and the second set of remaining landmarks by performing an image processing procedure on the alignment of the second set of remaining landmarks using a time warping procedure to adjust the temporal spacing of the landmarks in the second plurality of landmarks.

6. The apparatus of claim 1, wherein the at least one landmark of the first plurality of landmarks comprises a side branch junction point indicative of a location of a side vessel extending from a main artery.

7. The apparatus of claim 1, wherein the at least one landmark of the first plurality of the landmarks comprises a side branch junction point indicative of a location where a circumflex artery extends from a main artery.

8. The apparatus of claim 1, wherein co-registration further comprises enhancement the co-registration matching accuracy using a features-based correction algorithm.

9. The apparatus of claim 1, further comprising:
generating a first set of graphical components representing the first image frame with the first plurality of landmarks graphically indicated at their respective locations along a vessel as depicted in the first image frame;
generating a second set of graphical components representing the second image frames with the second plurality of landmarks graphically indicated at their respective locations along the vessel as depicted in the second image frames; and
generating a graphical image showing the co-registration.

10. The apparatus of claim 9, wherein generating the graphical image showing co-registration includes displaying the graphical image having the second plurality of landmarks graphically overlaid onto the graphical image of the first image frame.

11. The apparatus of claim 9, wherein generating the graphic image showing co-registration including displaying the graphical image comprises:
displaying the first image frame with the first plurality of landmarks graphically indicated at the respective locations along a vessel as the vessel is depicted in the first image frame; and
displaying the second image frames with the second plurality of landmarks graphically indicated at respective locations along the vessel as the vessel is depicted in the second image frame,
wherein the second image frames and the second plurality of landmarks are displayed in a parallel image alongside of the first image frame and the first plurality of landmarks, and arranged so that each of the first plurality of landmarks aligns with a corresponding one of the second plurality of landmarks, wherein corresponding landmarks represent a same physical feature along the vessel as imaged in both the first image frame and in the second image frames.

12. A non-transitory computer-readable storage device comprising instructions executable by a processor of a computing device, wherein the instructions, when executed by the processor cause the computing device to:
identify a first plurality of landmarks in a first image frame corresponding to a first imaging modality;
identify a second plurality of landmarks in a second image frame corresponding to a second imaging modality different from the first imaging modality;
match a first start point landmark designated in the first plurality of landmarks in the first image frame to a corresponding second start point landmark feature from the second plurality of landmarks; and
co-register the plurality of second image frames with the first image frame based on the matching, wherein the first imaging modality is coronary computed tomographic angiography and wherein the second imaging modality is intravascular ultrasound.

13. The non-transitory computer-readable storage device of claim 12, wherein the instructions when executed to match the first start point landmark designated in the first plurality of landmarks in the first image frame to the corresponding second start point landmark feature from the second plurality of landmarks cause the computing device further to:
align the first start point landmark designated in the first plurality of landmarks with the corresponding second start point landmark from the second plurality of landmarks; and
with the first start point landmark aligned with the second start point landmark, align each of a first set of remaining landmarks included in the first plurality of landmarks with a corresponding second set of remaining landmarks included in the second plurality of landmarks using linear mapping.

14. The non-transitory computer-readable storage device of claim 13, wherein the first start point landmark comprises a landmark corresponding to a side branch junction point where a left anterior descending (LAD) artery couples with a main artery.

15. The non-transitory computer-readable storage device of claim 13, wherein the first start point landmark comprises a landmark corresponding to a side branch junction point where a left circumflex (LCX) artery couples with a main artery.

16. The non-transitory computer-readable storage device of claim 13, wherein the instructions to co-register the plurality of second image frames with the first image frame based on the matching cause the computing device further to:
adjust the alignment between the first set of remaining landmarks and the second set of remaining landmarks by performing an image processing procedure on the alignment of the second set of remaining landmarks using a time warping procedure to adjust the temporal spacing of the landmarks in the second plurality of landmarks.

17. The non-transitory computer-readable storage device of claim 13, wherein the instructions, when executed by the processor, cause the computing device to:
generate a first set of graphical components representing the first image frame with the first plurality of landmarks graphically indicated at their respective locations along a vessel as depicted in the first image frame;
generate a second set of graphical components representing the second image frames with the second plurality of landmarks graphically indicated at their respective locations along the vessel as depicted in the second image frames; and
generate a graphical image showing the co-registration, wherein the generated the graphical image showing co-registration includes displaying the graphical image having the second plurality of landmarks graphically overlaid onto the graphical image of the first image frame.

18. The non-transitory computer-readable storage device of claim 17, wherein the generated graphical image showing co-registration is generated in real-time while an IVUS image capturing system coupled to the computing device is performing an imaging procedure on a patient.

19. A method for a cross-modality image co-registration system, the method comprising:
identifying, by a computing device, a first plurality of landmarks in a first image frame corresponding to a first imaging modality;
identifying, by the computing device, a second plurality of landmarks in a second image frame corresponding to a second imaging modality different from the first imaging modality;
matching, by the computing device, a first start point landmark designated in the first plurality of landmarks in the first image frame to a corresponding second start point landmark feature from the second plurality of landmarks; and
co-registering the plurality of second image frames with the first image frame based on the matching, wherein the first imaging modality is coronary computed tomographic angiography and wherein the second imaging modality is intravascular ultrasound.

20. The method of claim 19, wherein co-registering the plurality of second image frames with the first image frame based on the matching comprises:
aligning the first start point landmark designated in the first plurality of landmarks with the corresponding second start point landmark from the second plurality of landmarks; and
with the first start point landmark aligned with the second start point landmark, aligning each of a first set of remaining landmarks included in the first plurality of landmarks with a corresponding second set of remaining landmarks included in the second plurality of landmarks using linear mapping.
